(19) United States
(12) Patent Application Publication
(10) Pub. No.: US 2005/0069570 A1
(43) Pub. Date: Mar. 31, 2005

(54) Method of cell taking on surface of article with three-dimensional structure

(76) Inventors: Toshifumi Ishibashi, Ibaraki (JP); Tadao Ohno, Ibaraki (JP)

Correspondence Address: GREENBLUM & BERNSTEIN, P.L.C., 1950 ROLAND CLARKE PLACE, RESTON, VA, 20191, US

(21) Appl. No.: 10/494,518

(22) PCT Filed: Nov. 14, 2002
(86) PCT No.: PCT/JP02/11869

(30) Foreign Application Priority Data
Nov. 15, 2001 (JP) 2001-349614

(51) Int. Cl. C12N 5/08

(52) U.S. Cl. 424/423; 435/366

(57) ABSTRACT

A method for implanting cells derived from a living body as living cells onto a surface of a three-dimensional structure having a complicated configuration such as teeth, dental implants, artificial bones and artificial vessels, which comprises the steps of: (a) preparing a mold matching a configuration of a surface of the three-dimensional structure, and (b) introducing a suspension of the cells into the mold, and then fitting the three-dimensional structure into the mold for incubation, and a three-dimensional structure having a surface on which cells derived from a living body are implanted as living cells, which is obtainable by the method.

### TECHNICAL FIELD

The present invention relates to a method for implanting living cells derived from a living body onto a surface of three-dimensional structure. More specifically, the present invention relates to a method for implanting living cells derived from a living body onto a surface of three-dimensional structure such as artificial organs or artificial tissues, which are used by embedment in a living body or by equipment outside a living body. The invention also relates to a three-dimensional structure obtainable by said method.

### BACKGROUND ART

Among periodontal diseases, chronic inflammation caused by the infection with oral microorganisms around the paradentium is most frequently observed. As a result of the inflammation, the resorption of alveolar bone and gingival regression are caused, which finally results in deciduation of teeth. Further, with the periodontal diseases, a patient feels aches in the paradentium at every chew of food, and therefore, tooth extraction is often not avoidable. A therapy has so far been applied which is based on transplantation using heterogenous or autologous natural teeth, artificial teeth, dental implants or the like for the deciduous teeth or extracted teeth.

However, there are significant differences between natural tooth roots and artificial teeth or dental implants (artificial dental roots). Natural tooth roots are covered with periodontal membrane, and normally, the resorption of alveolar bone that supports the teeth is not observed. On the other hand, when a natural tooth after the removal of periodontal membrane is transplanted (Lang, H., et al., Formation of differentiated tissues in vivo by periodontal cell population cultured in vitro, J. Dent. Res., 74, pp.1219-25, 1995), or when a dental implant without periodontal membrane is transplanted (Takanori Eto, Difference in support mechanism and sensory level between implants and natural teeth, Tsuneo Suetsugu & Naoyuki Matsumoto Ed., Dental Implant, First Edition, Sentan Iryo Gijutsu Kenkyusho, Tokyo, pp.113-119, 2000), a problem arises in that the resorption of alveolar bone that supports the implants will be caused with the passage of long period of time, and finally the implants will become not usable. Therefore, it is considered to be a key of the transplantation of the tooth to perform the transplantation with periodontal membrane of the tooth to be transplanted as fresh as possible (Mitsuhiro Tsukiboshi, Practice of autologous transplantation of the tooth, Tsuneo Suetsugu & Naoyuki Matsumoto Ed., Dental Implant, First Edition, Sentan Iryo Gijutsu Kenkyusho, Tokyo, pp.247-251, 2000).

The periodontal membrane cells obviously contribute to the formation and maintenance of periodontal tissues (Kotaro Fujita, Odontogenic histology, Ishiyaku Shuppan, Tokyo, pp.159-190, 1981). If natural teeth contaminated with oral bacteria are subjected to an ordinary sterilization, periodontal membrane cells are also killed. Therefore, if periodontal membrane cells are exogenously implanted and allowed to survive on the tooth root of natural tooth, or the tooth root of an artificial tooth or dental implant that do not inherently have periodontal membrane, and if a periodontal membrane-like tissue closely similar to natural periodontal membrane tissue can be formed, long-term use of the transplant will become possible.

As a conventional method of attaching periodontal membrane cells, a method has been generally used which comprises the step of simply placing a tooth or an implant in a suspension of a large amount of periodontal membrane cells and incubated to expect attachment of living cells. However, microscopically, tooth roots of natural teeth have complicatedly curved surface structures, and for this reason, if periodontal membrane cells are suspended in an ordinary culture medium and poured over the tooth root of a natural tooth which is sand placed, most of the cells slide off the curved surfaces, and the cells can be implanted only a very limited area. The same problem occurs when dental implants are used.

Accordingly, many attempts have been made for the purpose of efficiently implanting living periodontal membrane cells in a large area on a three-dimensionally curved surface of a three-dimensional structure such as teeth and dental implants. For example, the report of Choi et al. (Choi, B. H., Periodontal membrane formation around titanium implants using cultured periodontal membrane cells, A pilot study, Oral Maxillofac Implants, 15, pp.193-196, 2000), the report of Kinoshita (Tomohiko Kinoshita, Shinichi Fukuoka, Takehiro Hidaka, Regeneration of periodontal membrane on artificial dental root, Tsuneo Suetsugu & Naoyuki Matsumoto Ed., Dental Implant, First Edition Sentan Iryo Gijutsu Kenkyusho, Tokyo, pp.305-311, 2000) and the method of Shimizu et al. (Japanese Patent Unexamined Publication (Kokai) No. 6-7381) are known.

Choi et al. collected periodontal membrane left on a tooth root of canine extracted tooth and finely sliced the membrane, and placed the slices directly on a surface of an implant, and then they incubated the implant until periodontal membrane cells migrated from the slices covered a large area of the surface of the implant to form a periodontal membrane-like tissue, and further they again transplanted the resulting periodontal membrane-like tissue to the same dog (the dog from which the extracted tooth was derived). As a result, they observed the formation of periodontal membrane and cementum on the surface and surround of the implant after 3 months. However, this technique has drawbacks in that, for example, a sufficient amount of periodontal membrane is first needed to be collected; if the periodontal membrane is contaminated with oral microorganisms, the membrane is required to be sterilized without killing the cells; and 4 to 5 weeks of culture is required until periodontal membrane cells, migrated from unevenly disposed periodontal membrane slices, sufficiently cover the surface of an implant, and thus considerable period of time is required.

Kinoshita et al. cultured periodontal membrane cells in a three-dimensional manner in a collagen gel and placed a collagen-immobilized implant in the gel to have the periodontal membrane cells incubated on the surface of the implant. However, according to the technique, the cells are maintained in the collagen gel to prevent the cells sinking under the gravity, and accordingly, attachment of the periodontal membrane cells to the implant surface is suppressed due to the anchorage dependency of the cells. Periodontal membrane cells away from the implant surface even by a small distance cannot adhere as living cells to the implant surface, which render the cell inoculation efficiency poor.

Shimizu et al. developed a method comprising the steps of culturing periodontal membrane cells in a three-dimensional manner in a collagen gel, impregnating the gel into atelocollagen sponge and further culturing the cells to form a layered culture sheet, and then winding the sheet around an artificial dental root. However, this method requires a technically complicated operation of winding and fixing the sponge (and occasionally further continuing the culture).

Therefore, all of the aforementioned attempts have problems, and an excellent technique has not yet been established which enables implanting of periodontal membrane cells as living cells onto a surface of a three-dimensional structure having a complicated configuration such as teeth and dental implants. Further, like tooth roots, there are substantially no artificial tissue or artificial organ for embedment in a living body or attachment to an outside surface of a living body having a wide and flat structure, on which cells can be deposited in a simple manner. As well as in the filed of dental materials, the aforementioned problem that no technique is available for satisfactorily implanting living cells on a surface of a three-dimensional structure having a complicated configuration is also found in the field of producing hybrid type artificial tissues and artificial organs constituted by artificial materials and cells derived from a living body.

### DISCLOSURE OF THE INVENTION

Thus, an object of the present invention is to provide a method for efficiently implanting living cells derived from a living body as living cells in a wide area onto a surface of a three-dimensional structure having a complicated configuration such as organs and tissues in living bodies.

Another object of the present invention is to provide a three-dimensional structure having a surface on which living cells derived from a living body are implanted.

The inventors of the present invention made various efforts to achieve the aforementioned objects. As a result, they found that:
(1) even for a tooth root having a complicated configuration, living cells can be implanted on the tooth root by preparing a mold that matches the configuration of the tooth root, introducing a small amount of a suspension of cultured periodontal membrane cells in the mold, then fitting the tooth root into the mold for incubation;
(2) when the mold consists of a material having little cytotoxicity and no cell-adhesive property, or the mold is subjected to a surface treatment with the aforementioned material, periodontal membrane cells can be efficiently implanted as living cells onto the tooth root;
(3) when small grooves and/or pores are provided on the internal surface of the mold and/or the surface of the tooth root, the suspension of periodontal membrane cells is not easily eliminated when the tooth root is fitted to the mold, and as a result, the suspension remains in the small grooves and/or the pores so that the cells can further efficiently be implanted as living cells onto the tooth root; and
(4) after the incubation according to the above (1), when the tooth root is taken off from the mold, and the tooth root is immersed in a culture medium and incubated again, the implanted living periodontal membrane cells will survive on the tooth root surface and expand to form a periodontal membrane-like tissue. The present invention was achieved on the basis of these findings.

The present invention thus provides a method for implanting living cells derived from a living body onto a surface of a three-dimensional structure, which comprises the steps of:
(a) preparing a mold which matches a configuration of the surface of the three-dimensional structure, and
(b) introducing a suspension of said cells into the mold, and then fitting the three-dimensional structure into the mold for incubation.

The present invention also provides a three-dimensional structure having a surface on which cells derived from a living body are implanted as living cells. This three-dimensional structure can be preferably produced by the aforementioned method.

According to preferred embodiments of the present invention, periodontal membrane cells can be implanted as living cells onto a wide area of the tooth root of a human extracted tooth, and similarly, periodontal membrane cells can be efficiently implanted as living cells onto an artificial tooth or a dental implant. In addition, it is also possible to allow the implanted living periodontal membrane cells to continuously survive to form a periodontal membrane-like tissue. This enables the prevention of resorption of alveolar bones that support transplanted teeth and dental implants, and the retention of teeth which are usable for a prolonged period of time. Accordingly, a treatment of a dental disease such as a periodontal disease becomes possible in which exodontia was unavoidably operated.

Further, according to preferred embodiments of the present invention, cells can be efficiently implanted as living cells onto a surface of an artificial tissue or an artificial organ having a complicated three-dimensional structure for embedment in a living body. In addition, it is also possible to allow the implanted living cells to continuously survive to produce hybrid-type artificial tissues and artificial organs having a tissue analogous to the tissue in a living body.

### BRIEF DESCRIPTION OF THE DRAWINGS

FIG. 1 shows a flowchart of the methods of forming a mold of a tooth root, adhering periodontal membrane cells and culturing the cells.

FIG. 2 includes photographs showing the results of follow-up culture of periodontal membrane cells on human tooth root surfaces.

A. A human tooth subjected to sterilization in a medium for transport containing gentamycin at a high concentration. After the alkaline phosphatase staining after the follow-up culture, uniform deposition of the azo dye in a dark bluish violet color is observed. This result indicates that periodontal membrane cells were implanted as living cells and sufficiently expanded on the surface of the tooth root.

B. A human tooth as a control which was stored in 70% alcohol and sterilized by autoclaving. No uniform deposition of the azo dye in a dark bluish violet color is observed. This result indicates that living periodontal membrane cells were not implanted.

### BEST MODE FOR CARRYING OUT THE INVENTION

The method for implanting living cells onto the surface of a three-dimensional structure of the present invention typically includes the following steps.

(1) Preparation of a Mold

In this step, a mold that matches the configuration of a three-dimensional structure is prepared.

The “three-dimensional structure” according to the present invention means a three-dimensional structure having a complicated configuration, specifically, an artificial organ, an artificial tissue or the like, and typical examples include artificial tooth roots.

According to the first embodiment, the mold itself consists of a material having little cytotoxicity and a property that living cells are hardly implanted. The mold can be prepared by, for example, adding a solution of the material, which has little cytotoxicity and the property that living cells are hardly implanted, to the surround of a three-dimensional structure and solidifying the material by cooling.

As the “material having little cytotoxicity and a property that living cells are hardly implanted”, for example, a material can be suitably used which has flowability before solidification, but is solidified by an appropriate treatment available for those skilled in the art and can exhibit the aforementioned properties after solidification. A type of the material is not particularly limited. Typical examples include agarose and agar. Further, if a material having high flowability before solidification, the material can be used for a three-dimensional structure having a more complicated configuration.

Concentration of the aforementioned material varies depending on the type of the material, and the concentration is not particularly limited. As for agarose, for example, 4% aqueous solution may be used.

In addition, as another embodiment, the mold may be subjected to a surface treatment with the aforementioned material. According to the aforementioned embodiment, a type of a material for forming the mold is not particularly limited. A plastic material which is a solution under heating and is solidified by cooling, e.g., polystyrene, can be used as the material to form the mold. For example, a melted solution of polystyrene is solidified by cooling around a three-dimensional structure, and then the material, which has little cytotoxicity and a property that living cells are hardly implanted, is coated on the surface of the solidified mold (surface to which the three-dimensional structure is contacted). Examples of the material include poly(2-hydroxyethyl methacrylate), polyethylene glycol, agarose and the like.

Further, when the material is coated on the mold surface, a thickness of the coating may be from about the thickness of one molecule to 0.1 mm. A concentration of a coating solution used for the coating is about 0.3% by weight, for example, when agarose is used as the coating agent.

Besides the aforementioned embodiment, any surface processing technique may be used so long as said technique can prevent living cells from implantation on a surface to be contacted with a three-dimensional structure, and provide a surface having little cytotoxicity.

The mold is generally formed by using a desired three-dimensional structure. When the configuration of a mold for a three-dimensional structure can be designed beforehand, the mold for a three-dimensional structure may be prepared by using a plastic block or a metal block having a property that living cells are hardly implanted, and the surface of the mold may be coated with the aforementioned material having the property that living cells are hardly implanted. Also in this embodiment, any surface processing technique may be applied so long as the technique provides a surface having little cytotoxicity and the property that living cells are hardly implanted.

In the present invention, small grooves and/or small pores may be provided on the mold surface prepared as described above so that the cell suspension can stay on the mold surface. The small grooves and/or the small pores may be provided by a method available for those skilled in the art such as a method of scratching the mold surface with a dental exploratory needle or analogous needle, or they may be provided any other methods, and the method is not particularly limited.

It may sufficient that the small grooves and/or the small pores have at least a size that enables cell invasion and can avoid significant deformation of the structure of the mold. For example, each of diameter and depth is preferably about 1 mm but not necessarily limited thereto. The number thereof can also be suitably selected. The grooves or the pores may preferably be present in a number as large as possible within a range that the shape of the mold is not significantly degraded.

By providing the small grooves and/or the small pores on the mold surface as described above, the cell suspension poured into the mold enters into those small grooves and/or the small pores, and when a three-dimensional structure is fitted into the mold, the three-dimensional structure does not entirely adhere to the mold, thereby the cell suspension can be prevented from being extruded and leaked from the mold. As a result, living cells can be quickly implanted onto the surface of the three-dimensional structure.

The aforementioned small grooves and/or the small pores may be provided on the surface of a three-dimensional structure. By providing the small grooves and/or the small pores on the surface of a three-dimensional structure, cells will invade into these small grooves and/or the small pores to facilitate implantation of living cells onto the surface of the three-dimensional structure.

In the embodiment, a material having threads or pores corresponding to the small grooves and/or the small pores, such as commercially available dental implants, may be used, per se. The surface of three-dimensional structure provided with the small grooves and/or the small pores is further coated with a material having a property that enables more easy implantation of cells, including cell adhesion factors such as collagen, fibronectin and laminin, thereby cell adhesion can be enhanced. The material and method used for the coating in this embodiment may be those available to those skilled in the art.

The aforementioned small grooves and/or the small pores may be provided either on the mold side or the three-dimensional structure side, or on the both sides, as required. If they are provided on the both of the mold side and the three-dimensional structure side, cells will invade into the small grooves and/or the small pores and temporarily held therein depending on the volumes of the small grooves and/or the small pores, and thus a state can be achieved that living cells are not implanted on the mold surface, whilst the cells are more likely implanted on the three-dimensional structure side.

(2) Culture for Implantation of Living Cells

When the mold in the above (1) is prepared by using a three-dimensional structure so as to be integrated with the three-dimensional structure, the mold is removed from the three-dimensional structure. When the mold can be independently designed, and the mold is separately prepared in a form independent from the three-dimensional structure, and the mold, per se, is used in the following step.

A separately prepared suspension of cells derived from a living body is introduced into the resulting mold, and a three-dimensional structure is fitted into the mold and incubated. As a result of this operation, the cells will be implanted onto the surface of the three-dimensional structure to which the living cells likely attach, but not onto the surface of the mold to which living cells can hardly attach. According to this procedure, it is unnecessary to enclose the cells in a collagen gel to prevent the sinking of the cells under the gravity, as in the aforementioned method of Kinoshita et al. and the cells suspended in a culture medium can be directly implanted onto the three-dimensional structure, without applying the complicated procedure of winding a layered culture sheet around the surface of an artificial tooth root as described in Shimizu et al. The number of cells required for this purpose may be less than that required for a procedure in which a mold is not utilized. However, the number of cells is not particularly limited. A number of cells may be sufficient that is expected to cover the three-dimensional structure in a desired area known to those skilled in the art after the follow-up culture described later.

In the present invention, the terminology “implantation of living cells” means that the cells are attached and fixed on an objective surface of the three-dimensional structure in a living state, and that the expanded cells exist densely and in a spread manner to form a tissue like a natural tissue of the cells, and it does not mean a state of the cells kept in simple non-densely adhesion to the surface of three-dimensional structure from the suspended state.

As the cells derived from a living body, cells may be preferably used which are suitable for a purpose of using a three-dimensional structure to which the cells are to be implanted. For example, when human periodontal membrane cells are implanted as living cells onto a human natural tooth for use in transplantation therapy, autologous human periodontal membrane cells of a patient to be treated are most preferred.

In the present invention, cells derived from various animals including human and cells derived from various tissues can be used as the cells derived from a living body. Examples include, for example, periodontal membrane cells, osteoblasts, chondrocytes, synovial cells, fibroblasts, vascular endothelial cells, cornea cells, lens cells, oral cavity mucous cells, pharynx epithelial cells, larynx epithelial cells, esophagus epithelial cells, bronchial epithelial cells, alveolar epithelial cells, hepatogenous cells, bile duct cells, gall bladder cells, kidney-derived cells, transitional epithelial cells, intestine mucous cells and the like.

The method for preparing the cell suspension used in the present invention is not particularly limited so long as a method enables maintained survival of the cells, and methods available to those skilled in the art may be used. Further, the condition for incubation of the three-dimensional structure after the fitting thereof into the mold is not particularly limited. For example, culture at 37° C. for 1 day is preferred. However, the incubation condition is not limited to the aforementioned condition, and any condition that enables implantation of living cells onto a three-dimensional structure surface may be used. The term “incubation” includes simple left standing of the culture.

As the cell culture medium, culture media available to those skilled in the art may be used. For periodontal membrane cells, for example, the RHAM α medium is most preferred which is obtained by adding supplements to the RHAM α (−) medium except interleukin-2 and anti-CD3 monoclonal antibody (Kawai, K. et al., Additive effects of antitumor drugs and lymphokine-activated killer cell cytotoxic activity in tumor cell killing determined by lactate-dehydrogenase-release assay, Cancer. Immunol. Immunother, 35, pp.225-229, 1992), further supplemented with fetal bovine serum up to a concentration of 10%(v/v). However, the medium is not particularly limited so long as a medium enables maintained living state of the human periodontal membrane cells, and any type of medium may be used. Period of time for the culture may also be optionally selected, and may preferably be 2 to 4 weeks. The period of time may also be determined according to a method available to those skilled in the art. According to a typical example, periodontal membrane cells expand sufficiently, even not perfectly, over a tooth root within 3 weeks, and the culture time may be shorter than the period of 5 to 6 weeks described in the aforementioned report of Choi et al. utilizing canine periodontal membrane (Choi, B. H., Periodontal membrane formation around titanium implants using cultured periodontal ligamnet cells, A pilot study, Oral Maxillofac Implants, 15, pp.193-196, 2000).

The three-dimensional structure onto which living cells are implanted by the method described above, such as a tooth or a dental implant, is removed from the mold and immersed in a culture medium that allows survival or proliferation of the cells to culture the cells on the three-dimensional structure surface and thereby form a tissue produced by the cells. The follow-up culture is preferably carried out on the three-dimensional structure surface, and conditions for the culture may be appropriately determined depending on the properties of the cells used and a purpose of a therapeutic use. For example, periodontal membrane cells implanted as living cells onto a tooth root expand, and sometimes proliferate, on the tooth root surface by the follow-up culture, and form a periodontal membrane-like tissue during the culture.

### EXAMPLE

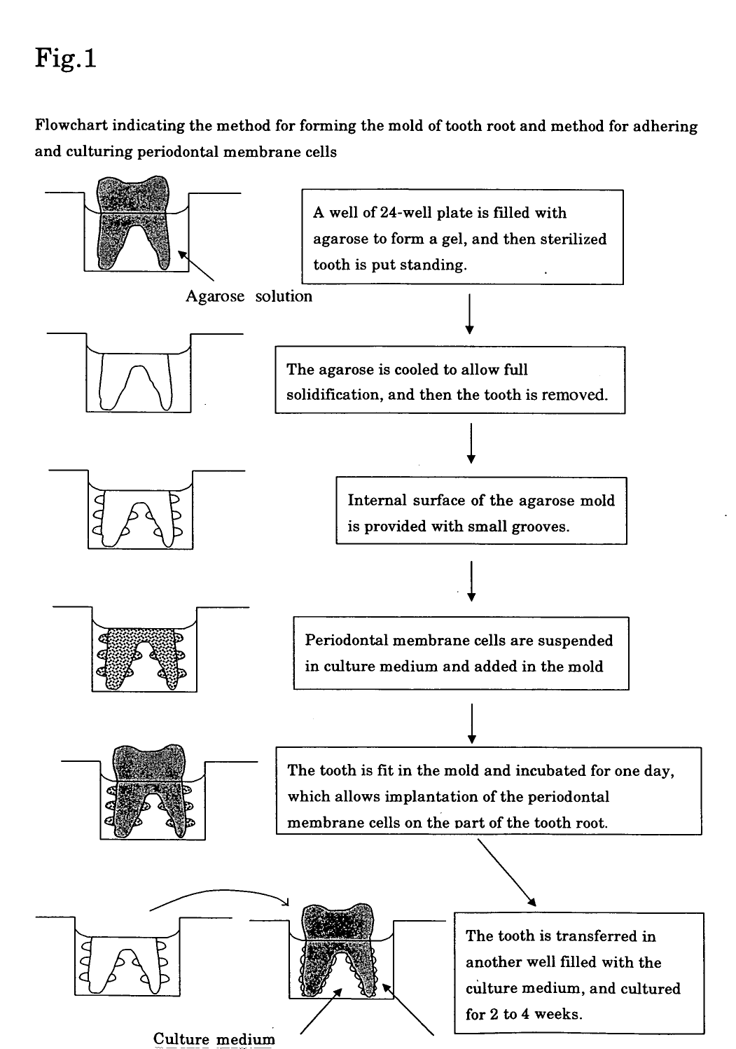

The present invention will be explained more specifically with reference to the following example. However, the scope of the present invention is not limited to the example. The method for implanting living cells of the present invention is outlined in FIG. 1 as a flowchart.

### Example 1

(1) Preparation of Periodontal Membrane Cells

Teeth with no inflammation in a clinical sense were used, which were extracted under informed consent from dental clinic outpatients who needed orthodontic therapy of impacted wisdom teeth or malpositioned teeth. Each extracted tooth was first washed with sterilized physiological saline to remove blood, and gingivae and dental calculi remained on the tooth neck were removed with sterilized scalpel and sterilized bar of dental turbine. The tooth was immediately placed in the culture medium for transportation cooled at 4° C. (Table 1)

TABLE 1
Culture medium for transportation
RHAM α (−) (mixed culture medium of commercially
available basal medium for animal cells RPMI1640,
HAM-F12, and MEM α at a mixing ratio of 3:1:1)

| | | |
|---|---|---|
| Additives | Gentamycin | 10 μg/ml |
| | Streptomycin | 100 μg/ml |
| | Kanamycin | 60 μg/ml |

An irrigation solution (Table 2) was prepared, 5 dishes having a diameter of 6 cm and containing 5 ml of the irrigation solution were placed in a row, and the tooth was moved successively from the leftmost dish to the rightmost dish with shaking by means of forceps to sufficiently wash the tooth.

TABLE 2
Irrigation solution
Dulbecco's phosphate-buffered saline (PBS(−))

| | | |
|---|---|---|
| Additives | Penicillin | 200 IU/ml |
| | Gentamycin | 10 μg/ml |
| | Streptomycin | 100 μg/ml |
| | Kanamycin | 60 μg/ml |
| | Amphotericin B | 2.5 μg/ml |

The culture medium RHAM a containing 10% (v/v) of fetal bovine serum was introduced into one well of a 6-well culture plate in a volume of 10 ml, and the washed tooth was gently placed in the well and sank in the medium and then incubated under said conditions. On the next day, the tooth was moved to the adjacent well filled with 10 ml of the culture medium. In a similar manner, the culture medium was changed every day for the initial three or four days, and after then, the half of the medium was changed. In the culture step, when no bacterial infection was successfully observed, and the periodontal membrane cells, which were detached from the tooth and outgrown on the culture surface of the well, proliferated in the well and reached confluent, the cells were treated with trypsin in a conventional manner and then subcultured in a 35-mm culture dish. From one to three dishes on which the cells proliferated, a cell suspension in which the cells were suspended in 1 to 2 ml of the culture medium was prepared in a conventional manner.

(2) Sterilization of Extracted Tooth

The medium for transportation in a volume of 10 ml was placed in a 15-ml volume test tube, and each tooth was stored separately in the medium. The tooth was washed with the irrigation solution in the same manner as that in the foregoing section, and then incubated in the same manner as that in the foregoing section. When bacterial infection was observed during this culture step, the tooth was immediately transferred to the medium for transportation and sterilized by addition of an aqueous solution of gentamycin at a high concentration (20 mg/ml) so as to be a final concentration of gentamycin at 100 μg/ml, and incubation was continued for one night or more. When bacterial infection was still observed during the following washing and culture steps, the aforementioned step was repeated with an increased concentration of gentamycin, and the tooth was used in the following step after confirmation of disappear of the bacterial infection. As a reference, a tooth stored in a 70% alcohol solution after exodontia was washed with PBS(−), sterilized by autoclaving at 120° C. for 20 minutes and then used.

(3) Implanting of Periodontal Membrane Cells onto Tooth and Culture of the Cells

Agarose (3 g) was added with 75 ml of water, warmed and dissolved by using a microwave oven. This 4% agarose solution obtained by the dissolution was put into each well of 24-well culture plate and left stand until it became like a gel state. In the agarose gel, each tooth sterilized and washed with PBS(−) was put standing and left until the agarose gel was solidified to prepare a mold matching the configuration of the tooth. Subsequently, the tooth was taken out, cleaned by rubbing the surface with sterilized forceps and immersed in a fibronectin solution (obtained by dissolving fibronectin in PBS(−) at a concentration of 10 μg/ml) for 1 or 2 days at room temperature. Small grooves were formed on the internal surface of the mold matching the configuration of the tooth with forceps or exploratory needle, and then an appropriate amount of the periodontal membrane cell suspension was poured into the mold matching the configuration of the tooth. Then, the tooth treated with fibronectin was fit stand and the culture medium was poured up to the height of the crown of tooth, and the tooth was incubated for 1 day. This tooth was transferred to another empty well, added with the culture medium and incubated for 2 to 4 weeks.

(4) Alkaline Phosphatase Staining

If the periodontal membrane cells survived on the surface of the incubated tooth after the aforementioned process, alkaline phosphatase activity derived from the cells exists, and deposit of the azo dye in a dark bluish violet color can be observed. Alkaline phosphatase staining was performed according the following procedure. First, the incubated tooth was immersed in 99.5% ethanol to fix the cells and washed 5 or 6 times with purified water. This tooth was immersed in the alkaline phosphatase reaction solution (Table 3), and the reaction was carried out at room temperature for about 30 minutes. The tooth was sufficiently washed with tap water, then stained with 1% methyl green nuclear staining solution (hematoxylin, Koeln Echte Roth) for 10 minutes, washed with tap water and purified water, and then dried.

TABLE 3
Alkaline phosphatase reaction solution

| | |
|---|---|
| Naphthol AS-BI (AS-MX) phosphoric acid | 5 mg |
| (disodium salt) (Sigma, Catalog | |
| number N2125) | |
| 2-Amino-2-methyl-1,3-propandiol | 10 ml |
| buffer (0.05 M, pH 9.8) | |
| Fast blue RR salt (Sigma, Catalog | 5 mg |
| number F0500) | |

(5) Results

As for the extracted tooth which was free from bacterial infection during the preparation of the periodontal membrane cells, when a culture plate which contained the tooth placed in the well was left stand in an incubator for several days, the proliferation of the cells was observed on the well surface of the culture plate. It is known that cultured periodontal membrane cells are different from cells of other periodontal tissues such as osteoblasts in morphological characteristics (Masahiro Kubota, Investigation relating to proliferation and function of periodontal membrane cell and osteoblast under hypoxia, Ku Byo Shi, 56, pp.473-484, 1989). The cells with active proliferation that were successfully cultured in the aforementioned experiment were found to be uniform fibroblast-like cells having a spindle shape with a large long axis under optical microscopy, and when they are subjected to proliferation, they also showed a characteristic of fibroblasts that the cells align along a certain direction. In addition, it was indicated from the alkaline phosphatase staining that these cells had the alkaline phosphatase activity. On the basis of the above two facts, the cells were identified as periodontal membrane cells. When these periodontal membrane cells were subcultured, they were subcultured for 5 to 10 generations with split of about 1:2 to 1:3. These periodontal membrane cells, which were able to be subcultured, was successfully cryopreserved and recultured after thawing in a conventional manner. Accordingly, they were usable in the experiment of reattachment on a sterilized tooth.

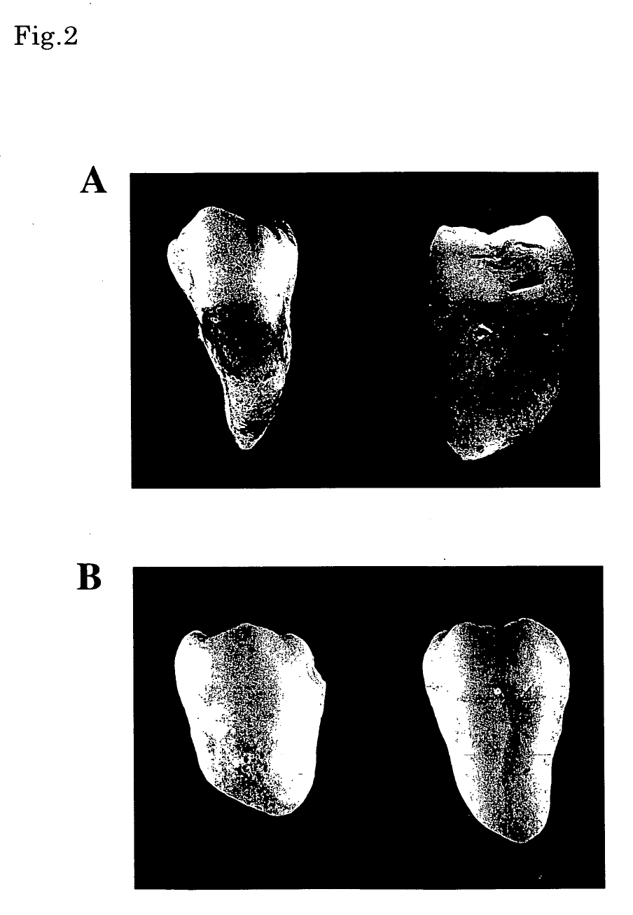

On the tooth sterilized in the medium for transportation containing gentamycin at a high concentration in the above (2), periodontal membrane cells separately cultured and prepared were implanted as living cells, and the cells were observed to be present on the tooth root surface through alkaline phosphatase staining after the follow-up culture (FIG. 2A). Judging from the fact that there was a continuously spreading region stained in a dark color, it was considered that the periodontal membrane cells did not simply attached non-densely from the suspended state, but that the expanded cells existed densely in a spread state to form a periodontal membrane-like tissue.

As for the tooth stored in 70% alcohol and subjected to sterilization by autoclaving used as the control, no uniform deposit of the azo dye in a dark bluish violet color was observed, and this result indicated that the periodontal membrane cells were not implanted as living cells onto the tooth (FIG. 2B). It is considered that this failure was caused because the cell adhesion factors on the tooth surface were denatured due to the storage in 70% alcohol and sterilization by autoclaving, and the periodontal membrane cells were not able to be implanted as living cells onto the tooth.

Industrial Applicability

According to the present invention, a method is provided for broadly and efficiently implanting cells derived from a living body as living cells onto a surface of a three-dimensional structure having a complicated configuration such as teeth, dental implants, artificial bones, and artificial blood vessels. A three-dimensional structure on which living cells derived from a living body are implanted, which can be prepared by the aforementioned method, has high biocompatibility as an artificial organ or artificial tissue used for embedment in a living body or equipment outside a living body, and thus enables effective therapy.

1. A method for implanting cells derived from a living body onto a surface of a three-dimensional structure as living cells, which comprises the steps of:
(a) preparing a mold matching a configuration of a surface of the three-dimensional structure, and
(b) introducing a suspension of the cells into the mold, and then fitting the three-dimensional structure into the mold for incubation.

2. The method according to claim 1, wherein the mold consists of a material having little cytotoxicity and a property that living cells can hardly be implanted, or the mold is subjected to a surface treatment with said material.

3. The method according to claim 1, wherein small grooves and/or small pores in which the suspension of the cells can be retained are provided on a surface of the mold and/or a surface of the three-dimensional structure.

4. The method according to claim 1, wherein the material is agarose, poly(2-hydroxyethyl methacrylate), or polyethylene glycol.

5. The method according to claim 1, wherein the cells derived from a living body are selected from the group consisting of periodontal membrane cells, osteoblasts, chondrocytes, synovial cells, fibroblasts, vascular endothelial cells, cornea cells, lens cells, oral cavity mucous cells, pharynx epithelial cells, larynx epithelial cells, esophagus epithelial cells, bronchial epithelial cells, alveolar epithelial cells, hepatogenous cells, bile duct cells, gall bladder cells, kidney-derived cells, transitional epithelial cells, and intestine mucous cells.

6. The method according to claim 1, wherein the three-dimensional structure is selected from the group consisting of a tooth, dental implant, bone, artificial joint, fastening stop, artificial ligament, artificial dura mater, artificial vessel, artificial cornea, intraocular lens, artificial larynx, artificial pharynx, artificial esophagus, artificial trachea, artificial lung, artificial chest wall, artificial breast, artificial heart, artificial flap, artificial pericardial sac, artificial diaphragm, artificial liver, artificial bile duct, artificial kidney, artificial bladder, artificial urinary duct, artificial pancreas, artificial abdominal wall, artificial intestine, artificial penis and artificial testicle.

7. A three-dimensional structure having a surface on which cells derived from a living body are implanted as living cells, which is obtainable by the method according to claim 1.
